(19) United States
(12) Patent Application Publication
(10) Pub. No.: US 2025/0255734 A1
(43) Pub. Date: Aug. 14, 2025

(54) SYSTEM AND METHOD FOR RESISTANCE CONTROL OF A MICROPROCESSOR- CONTROLLED PROSTHETIC KNEE

(71) Applicant: College Park Industries, Inc., Warren, MI (US)

(72) Inventors: Anna Mae Marlatt, Waterford, MI (US); Jacob Michael Drews, Washington, MI (US); Aaron John Taszreak, China, MI (US)

(21) Appl. No.: 19/046,946

(22) Filed: Feb. 6, 2025

Related U.S. Application Data
(60) Provisional application No. 63/551,811, filed on Feb. 9, 2024.

(51) Int. Cl. A61F 2/64; A61F 2/70; A61F 2/72; A61F 2/74; A61F 2/76

(52) U.S. Cl. CPC A61F 2/64; A61F 2/72; A61F 2/74; A61F 2/76; A61F 2002/704; A61F 2002/7625; A61F 2002/7635

(57) ABSTRACT

A prosthetic knee uses a hydraulic damper to regulate the rotation of the prosthetic knee joint. An IMU sensor detects the direction of rotation, tilt, and/or speed of the prosthetic knee. A magnetic rotary on-axis position sensor located at the joint between the upper and lower portions of the prosthetic knee measures the motion of the upper portion of the knee by detecting the magnetic field which is generated by a diametrically polarized magnet. A microprocessor correlates the measured motion to a knee joint angle. The microprocessor detects a user's stage of gait based on the measurements of the IMU sensor and on-axis position sensor and adjusts the resistance provided by the hydraulic damper according to a corresponding stage of gait.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims priority benefit to U.S. Provisional Application No. 63/551,811, filed Feb. 9, 2024, the entirety of which is hereby incorporated by reference herein.

### BACKGROUND

### Field

The present disclosure relates generally to a sensor system and control algorithm for regulating the rotation of a prosthetic joint, more specifically, a microprocessor controlled prosthetic knee joint.

### Description of the Related Art

Prosthetics are used to replace and restore the functionality of amputated natural body parts. Microprocessor-controlled prosthetic knees may use gearmotors for controlling the rotation of hydraulic valves. Present microprocessor-controlled prosthetic knees use a variety of methods for measuring the knee joint angle.

Some existing products measure the angle of the knee joint in order to determine modifications to the knee joint resistance depending on phase of gait and activity. In these devices, the upper bone, or thigh segment, pivots about an axial pin which connects the upper bone segment to the knee frame, lower bone, or shin segment. The shaft of the axial pin is a horizontal track which remains stationary during knee flexion and extension. A small magnet sits in said track. A hall effect sensor may be positioned on a circuit board at the front of the upper bone segment which measures the displacement of the magnet along the horizontal track. This displacement is correlated to an angle of the knee joint. This method is not ideal for determining the knee joint angle as it doesn't measure the angle directly at the source of rotation and it requires additional componentry. Furthermore, it requires additional moving parts, and the magnet can become loose from the track. Further, if debris ingresses into the system, it will impact the magnet as it travels along the track. If the magnet can no longer readily slide along the track, the knee joint angle will no longer be detectable. When the knee angle is no longer detectable, the knee will no longer function or trigger properly.

Other existing devices use an induction sensor for determining the knee joint angle. Instead of measuring the displacement of a magnet along a track, the upper bone portion of these prosthetic knees are made of a metal material of variable thickness surrounding the axial pin. An induction sensor is positioned in front of the upper bone segment, and, as the upper bone rotates about the axial pin, the thickness of the material between the upper bone and axial pin varies and the induction sensor can detect the amount of material in proximity. This detected thickness of the metal material is correlated to the knee joint angle. While this method has the added advantage of measuring the knee joint angle directly from the pivot point and can lessen the power requirements needed, it is not ideal because that induction sensors are generally less accurate than position or displacement sensors.

In other known devices, a diametrically polarized magnet is located on the axial pin and a linear Hall Effect sensor is positioned in front of the upper bone segment. As the knee joint moves, the Hall Effect sensor detects the magnetic field presence. This magnetic field is correlated to a knee angle which is used for making decisions on the joint resistances. However, the use of linear Hall effect sensors in such devices are not ideal as they are limited to measuring only linear motion.

In other known devices, an algorithm may be used to adjust the flexion resistance of a prosthetic knee based on measurements from knee angle and/or position sensors and inertial sensors (e.g., an inertial measurement unit). Such algorithms generally require the measurement of angles based on center of mass and chord length of the prosthesis. Additionally, such algorithms generally require multiple operating states for the various activities of daily living (ADLs) a wearer might experience including ramp and stairs modes.

### SUMMARY

The embodiments disclosed herein each have several aspects, no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices, and methods a prosthetic device, for example a microprocessor-controlled prosthetic knee.

The following disclosure describes non-limiting examples of some embodiments. Other embodiments of the disclosed systems and methods may or may not include the features described herein. Moreover, disclosed advantages and benefits can apply only to certain embodiments of the invention and should not be used to limit the disclosure.

In some aspects, the techniques described herein relate to a prosthetic joint, which may include: a frame; a connector pivotally coupled to a proximal portion of the frame, the connector configured to pivot in an anterior-posterior direction of the frame about a pivot axis that extends in a medial-lateral direction of the frame, the frame and connector being portions of a joint; a shaft extending along the pivot axis, the shaft extending through the connector and coupled to the frame on opposite sides of the connector; an inertial sensor attached to the frame, a knee angle sensor attached to the frame at or proximate the pivot axis; a microprocessor configured to receive signals detected by the inertial sensor to detect a gait event and the knee angle sensor and to automatically adjust a flexion and/or extension resistance of the prosthetic joint in response to the detected gait event; wherein the flexion and/or extension resistance of the prosthetic joint is controlled solely by information from the signals for the gait event detected by the knee angle sensor and the inertial sensor.

In some aspects, the techniques described herein relate to a prosthetic joint, wherein the prosthetic joint is a prosthetic knee.

In some aspects, the techniques described herein relate to a prosthetic joint, wherein the microprocessor adjusts the flexion resistance to a lower resistance level in response to the signals from the inertial sensor indicating the prosthetic joint is fully or hyper-extended, there is not an active backward rotation of the prosthetic joint, and there is a forward tilt of the prosthetic knee.

In some aspects, the techniques described herein relate to a prosthetic joint, wherein the microprocessor adjusts the flexion resistance to a lower resistance level only when: a detection is made by the knee angle sensor that the prosthetic joint is fully or hyper-extended; a detection is made by the inertial sensor that there is an active forward rotation of the prosthetic joint; a detection is made by the inertial sensor that there is a forward tilt of the prosthetic knee.

In some aspects, the techniques described herein relate to a prosthetic joint, wherein the prosthetic knee includes a hydraulic damper including a cylinder and piston configured for slidable travel within the cylinder.

In some aspects, the techniques described herein relate to a prosthetic joint, wherein the microprocessor is configured to control operation of the hydraulic damper in order to adjust a flexion resistance of the prosthetic joint.

In some aspects, the techniques described herein relate to a prosthetic joint, wherein the inertial sensor is an IMU.

In some aspects, the techniques described herein relate to a prosthetic joint, wherein the IMU includes one or more of: an accelerometer; a gyroscope; and/or a magnetometer.

In some aspects, the techniques described herein relate to a prosthetic joint, further including: a diametrically polarized magnet housed in a magnet cup, the magnet cup being housed in a hollow portion of the shaft so that the magnet is centered along the pivot axis, the magnet cup separating the magnet from the shaft, wherein rotation of the connector relative to the frame simultaneously rotates the shaft, the magnet cup, and the diametrically polarized magnet, and wherein the knee angle sensor is configured to measure a motion of the connector relative to the shaft by detecting a magnetic field generated by the magnet.

In some aspects, the techniques described herein relate to a prosthetic joint, wherein the magnet cup is configured to separate the diametrically polarized magnet from the shaft to not inhibit a magnetic field output of the diametrically polarized magnet.

In some aspects, the techniques described herein relate to a prosthetic joint, wherein the inertial sensor is attached to first circuit board.

In some aspects, the techniques described herein relate to a prosthetic joint, wherein the knee angle sensor is attached to a second circuit board.

In some aspects, the techniques described herein relate to a prosthetic joint, wherein the knee angle sensor is encased in a coating and/or an overmold and wherein the coating and/or the overmold is configured to electrically isolate the second circuit board.

In some aspects, the techniques described herein relate to a prosthetic joint, wherein the microprocessor is configured to correlate the signals from the knee angle sensor to a knee angle measurement.

In some aspects, the techniques described herein relate to a method for controlling a prosthetic joint, the method including: measuring, by an inertial sensor a first set of parameters of the prosthetic joint; measuring, by a knee angle sensor, a second set of parameters of the prosthetic joint; receiving, by a microprocessor, signals representing the first and second set of parameters; determining, by the microprocessor, an appropriate level of flexion and/or extension resistance to be applied to the prosthetic joint based on the signals; detecting, by the microprocessor, a gait event based on the received signals; and adjusting, by the microprocessor, a resistance of the prosthetic joint to the appropriate level of flexion resistance for the detected gait event solely based on the signals.

In some aspects, the techniques described herein relate to a method, wherein the prosthetic joint is a prosthetic knee.

In some aspects, the techniques described herein relate to a method, wherein the determining includes determining that one or more preconditions exist.

In some aspects, the techniques described herein relate to a method, wherein the one or more preconditions include one or more of: a detection that the prosthetic joint is fully or hyper-extended; a detection that there is an active forward rotation of the prosthetic joint; a detection that there is a forward tilt of the prosthetic knee.

In some aspects, the techniques described herein relate to a method, wherein the one or more preconditions include one or more of: a detection by the knee angle sensor that the prosthetic joint is fully or hyper-extended; a detection by the inertial sensor that there is an active forward rotation and/or not an active backwards rotation of the prosthetic joint; a detection by the inertial sensor that there is a forward tilt of the prosthetic knee.

In some aspects, the techniques described herein relate to a method, further including determining that at least one of the one or more preconditions do not exist; and adjusting, by the microprocessor, the flexion resistance to a first level.

In some aspects, the techniques described herein relate to a method, further including determining that each of the one or more preconditions exist; and adjusting, by the microprocessor, the flexion resistance to a second level.

In some aspects, the techniques described herein relate to a method, wherein the second level is lower than the first level.

In some aspects, the techniques described herein relate to a method, further including determining, by the microprocessor, that an unknown condition exists and adjusting and/or maintaining, by the microprocessor, the flexion resistance to/at the first level.

In some aspects, the techniques described herein relate to a method, wherein the one or more preconditions includes three preconditions.

In some aspects, the techniques described herein relate to a method, wherein the inertial sensor is an IMU.

In some aspects, the techniques described herein relate to a method, wherein the IMU includes one or more of: an accelerometer; a gyroscope; and/or a magnetometer.

In some aspects, the techniques described herein relate to a method, wherein the first set of parameters includes one or more of direction of rotation, tilt, and/or speed.

In some aspects, the techniques described herein relate to a method, wherein the microprocessor is configured to correlate the signals representing the second set of parameters to a knee angle measurement.

In some aspects, the techniques described herein relate to a method, further including initializing a user's initial vertical position.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

The following drawings are for illustrative purposes only and show non-limiting embodiments. Features from different figures may be combined in several embodiments.

FIG. 1 shows an example side view of a prosthetic knee.

FIG. 2 is an example side view of the axial pin shaft.

FIG. 3A shows an example of a knee angle circuit board.

FIG. 3B shows a close-up view of the knee angle circuit board of FIG. 3A.

FIG. 4A shows a schematic of how an example control algorithm may adjust the flexion resistance of the prosthetic knee during gait.

FIG. 4B shows another schematic of how an example control algorithm may adjust the flexion resistance of the prosthetic knee during gait.

FIG. 5 shows a schematic of how an example control algorithm may adjust the extension resistance of the prosthetic knee during gait.

FIG. 6 shows a front view of an example hydraulic damper of the prosthetic knee.

### DETAILED DESCRIPTION

The following detailed description is directed to certain specific embodiments of prosthetic devices and methods. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. The embodiments, examples of which are illustrated in the accompanying drawings, are set forth in detail below. Wherever possible, the same reference numbers are used throughout the drawings to refer to the same or like parts.

Microprocessor-controlled hydraulic prosthetic knees are an ideal solution for controlling the rotation of the prosthetic knee joint for above knee amputees. These systems employ one or more microprocessors and various sensors that detect, respond, and react to the bending of the knee joint by modifying the hydraulic resistance in both flexion and extension directions.

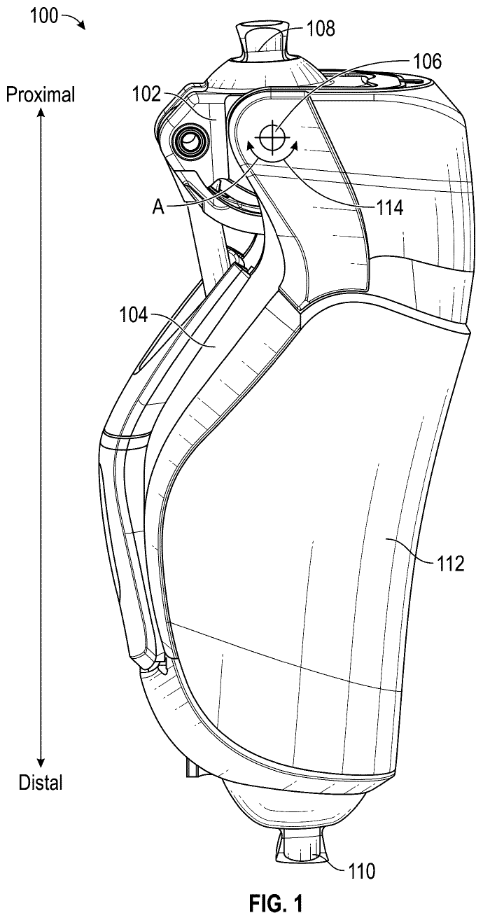

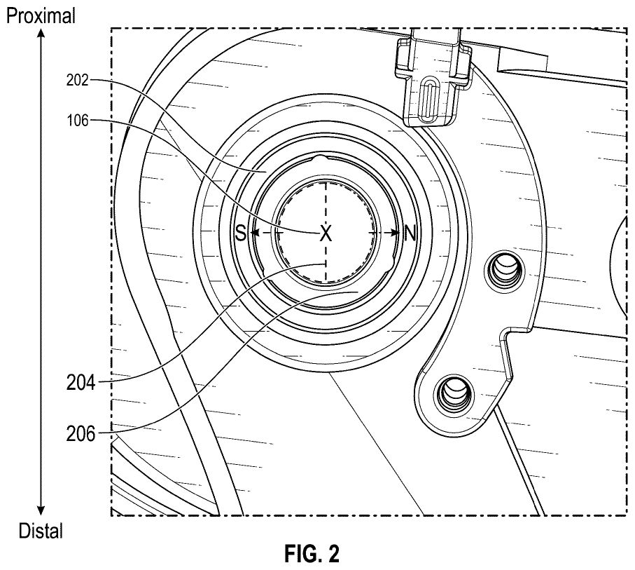

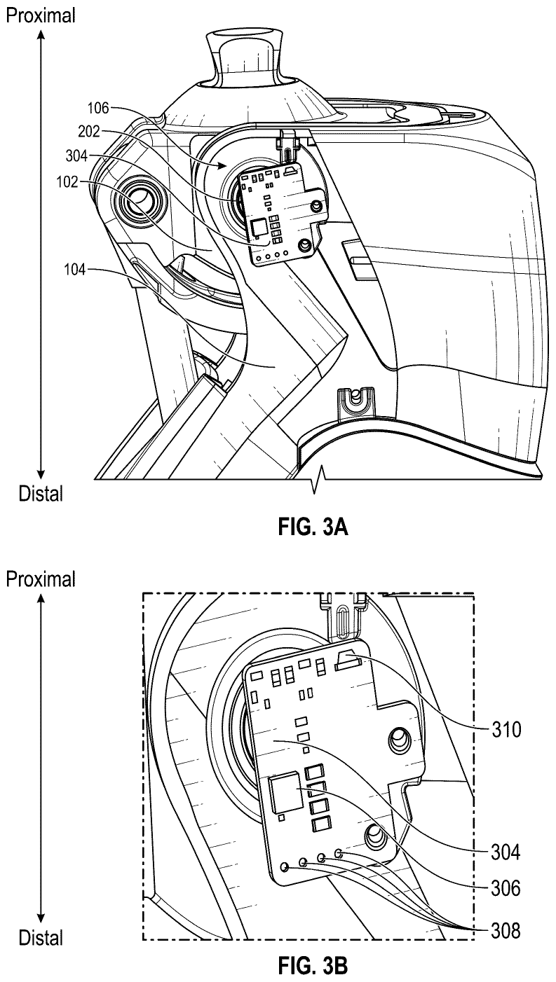

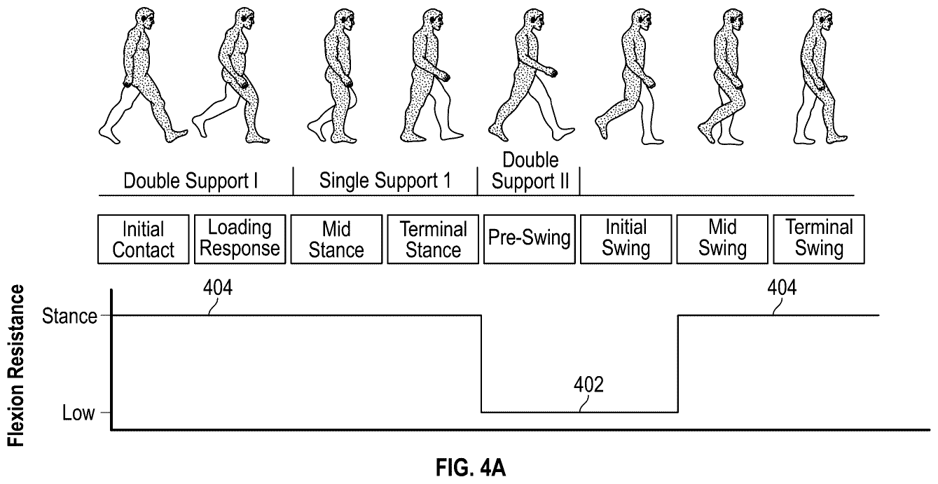

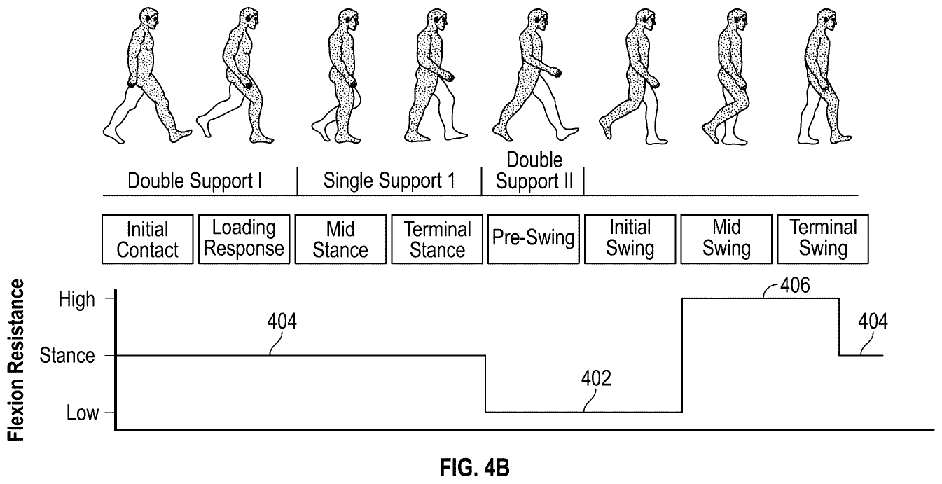

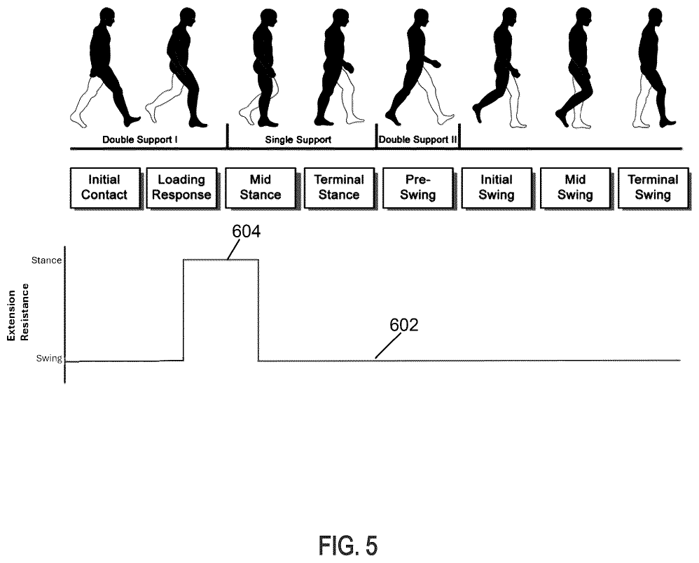

FIG. 1 shows an example side view of a prosthetic knee 100. The proximal end of the prosthetic knee 100 includes an upper frame portion 102, and the distal end of the prosthetic knee 100 includes and a lower frame portion 104. The upper frame portion 102 may include a proximal connector 108 (e.g., pyramid connector) at a proximal end for connecting the prosthetic knee 100 to an upper leg prosthetic device (e.g., a socket worn by an amputee over their upper leg). The lower frame portion 104 may include a distal connector 110 (e.g., pyramid connector) at a distal end for connecting the prosthetic knee to a lower leg prosthetic device (e.g., a pylon that couples to a prosthetic foot, a prosthetic foot, etc.). The prosthetic knee 100 may further include a hydraulic damper 500 housed within the body 112 of the lower frame portion 104, as described in more detail with respect to FIG. 5. The upper frame portion 102 and lower frame portion 104 are pivotably connected by a joint portion 114 to create a joint allowing for lateral and medial rotation about a pivot point 106 (i.e., or pivot axis, in the direction shown by arrow A). The joint portion 114 connects the upper frame portion 102 and the lower frame portion 104 at the pivot point 106. An axial pin shaft 202, as shown in FIGS. 2-3A, extends along the lateral axis of pivot point 106, through the joint portion 114 and is coupled to opposite sides (i.e., medial and lateral) of the lower frame portion 104 (e.g., allowing the upper frame portion 102 to pivot about the pivot point or pivot axis 106). The rotation of the upper frame portion 102 about the pivot point or pivot axis 106 is regulated by the hydraulic damper 500. To determine when to make changes to the bending (e.g., flexion and/or extension) resistance of the prosthetic knee 100, a knee angle sensor (not shown) may be used to determine the state of the prosthetic knee 100 during gait (e.g., during different phases of gait) and the bending resistance provided by the hydraulic damper 500 can be adjusted accordingly. As further described herein, additional sensors may be required in order to confirm changes that should be made to the bending resistance of the prosthetic knee 100. In some embodiments, as described further with respect to FIGS. 4A and 4B below, a control algorithm may use measurements from the knee angle sensor and one or more inertial sensors to detect a user's stage of gait, and accordingly instruct the microprocessor to automatically adjust the hydraulic damper 500 in order to provide an appropriate level of flexion and/or extension resistance to a user. Advantageously, control of flexion and/or extension resistance for the prosthetic knee 100 can be achieved with only the knee angle sensor and one or more inertial sensors, and without user-specific information.

FIG. 2 is an example side view of the axial pin shaft 202. The axial pin shaft 202 may be made of metal (i.e., steel, or other suitable metals). A diametrically polarized magnet 204 is housed within an axial pin magnet cup 206 inserted into a hollow portion of the axial pin shaft 202 such that the magnet 204 is centered over the pivot point 106. As the upper frame portion 102 of the prosthetic knee 100 rotates, the axial pin shaft 202 and magnet 204 inside of the axial pin magnet cup 206 will also rotate (e.g., simultaneously). The axial pin magnet cup 206 advantageously separates the magnet 204 from the axial pin shaft 202 so as to not inhibit the magnetic field output of the magnet 204. The axial pin magnet cup may be made of any non-magnetic metal or plastic such as, for example, aluminum or nylon.

FIG. 3A shows an example of a knee angle circuit board 304 which is disposed between the axial pin shaft 202 and the joint portion 114. FIG. 3B is an example close up view of the knee angle circuit board 304. The knee angle circuit board 304 may include one or more of a knee angle sensor (not shown) and optionally one or more (e.g., one, two, three, four, etc.) electrical or solder connections 308. Solder connections 308 may be used to connect the knee angle circuit board 304 to a power source (e.g., an internal battery located in the prosthetic knee 100), ground, and a main circuit board of the prosthetic knee 100. The main circuit board may be located at another location of the prosthetic knee 100 (e.g., on a ventral or dorsal side of the body 112) and may include, for example, one or more various sensors, a microprocessor, a Bluetooth module, and/or one or more inertial sensors or an inertial measurement unit (IMU).

The knee angle sensor (not shown) may be disposed on a back side of the knee angle circuit board 304 that faces the axial pin shaft 202. The knee angle sensor may be magnetic rotary position Hall Effect sensor, or any magnetic sensor capable of detecting magnetic field rotation. The knee angle sensor may be positioned directly in line with the pivot point 106 above the magnet 204 (e.g., on an inward-facing side of the knee angle circuit board 304) and be capable of measuring the motion of the upper frame portion 102 of prosthetic knee 100 by detecting the magnetic field generated by the magnet 204 during gait (e.g., during rotation of the knee joint). This motion can be correlated to a knee joint angle. The knee joint angle data is transferred from the knee angle sensor to the microprocessor of the main circuit board of the prosthetic knee 100 via, for example, solder connections 308.

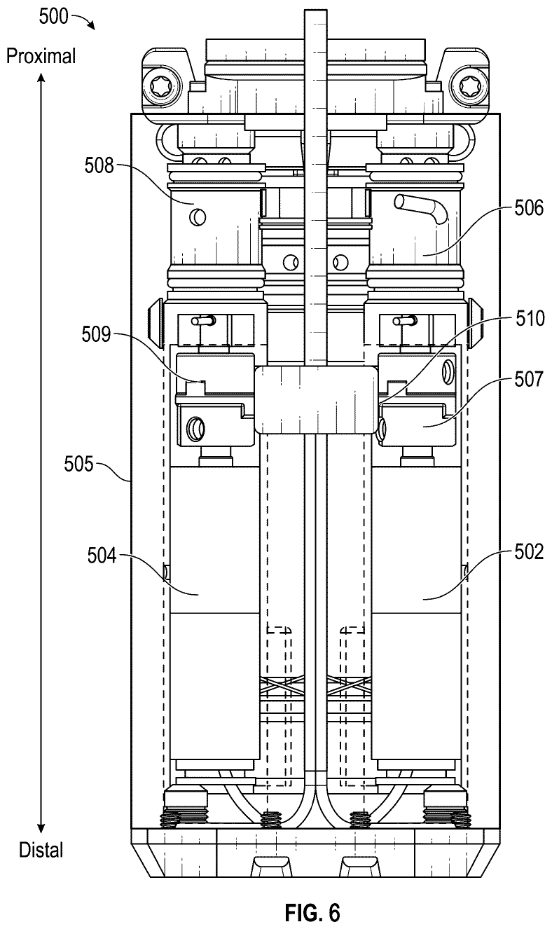

A control algorithm may be capable of recognizing gait patterns from the information received from the knee angle sensor and one or more inertial sensors, and various other sensors that may be included in the main circuit board of the prosthetic knee 100. The control algorithm reacts at transition points in the gait cycle by instructing the microprocessor to activate a gearmotor 502, 504 which adjusts a valve assembly in the hydraulic damper 500, as described in more detail below with respect to FIG. 6, to adjust flexion and/or extension resistance for the prosthetic knee 100.

In some embodiments, the knee angle circuit board 304 may be encased in an enclosure, coated (e.g., with Parylene C), or over-molded (e.g., in a resin) to protect it from environmental effects and impacts occurring while the prosthetic knee 100 is used or handled. The enclosure, coating or overmold may also advantageously provide electrical isolation of the knee angle circuit board 304.

In some embodiments, the knee angle circuit board 304 may further include a light emitting diode (LED) 310 that may illuminate (e.g., in different patterns or colors) to convey alerts to the use of the prosthetic. The knee angle circuit board 304 may be attached to the side of the lower frame portion 104 of the prosthetic knee 100 at the pivot point 106 between the upper frame portion 102 and lower frame portion 104 of the prosthetic knee 100.

In some embodiments, the microprocessor may execute instructions from a control algorithm to automatically adjust a flexion resistance of the prosthetic knee 100 during gait, as shown in FIGS. 4A and 4B. By default, the prosthetic knee 100 may provide high flexion resistance to support an end user. The control algorithm may determine when to reduce or increase the flexion resistance, and accordingly, instruct the microprocessor to activate a gearmotor 502, 504, which adjusts a valve assembly in a hydraulic damper 500, as described in more detail with respect to FIG. 6, to adjust knee flexion resistance. For example, when the control algorithm determines that a user is in a “Pre-Swing” or “Toe-Off” phase of gait (e.g., about to take a step), the control algorithm may instruct the microprocessor to adjust a valve assembly in a hydraulic damper 500 to reduce flexion resistance, allowing for heel rise (or swing flexion). When the control algorithm determines that a user is no longer achieving heel rise, the control algorithm may instruct the microprocessor to adjust a valve assembly in a hydraulic damper 500 to increase flexion resistance to provide sufficient loading support to the user. The control algorithm may continually run. A baseline resistance level (e.g., stance flexion level 404, stance flexion resistance level while knee is loaded), may be set by the Certified Prosthetist/Orthotist (CPO) on an end user basis through a control application available on smart devices.

The control algorithm may only require measurements from one or more inertial sensors (not shown), for example located on the main circuit board and the knee angle sensor in order to operate. In such embodiments, the main circuit board may be located on the dorsal side of the prosthetic knee 100. The algorithm may use the measurements of the one or more inertial sensors to detect one or more of the direction of rotation, tilt, and/or speed of the prosthetic knee 100 during gait. In some embodiments, the one or more inertial sensors may be an inertial measurement unit (IMU). The IMU sensor may contain both a three-axis accelerometer and three-axis gyroscope (e.g., the IMU sensor may be a six-axis IMU). The accelerometer may be used to measure the overall tilt of the knee. In some embodiments, a user's initial vertical position may be calibrated when they are initially fitted with the prosthetic knee 100 and then the accelerometer may be used to detect deviations from this initial position. The gyroscope may be used to measure the active angular acceleration (i.e., active rotation) of the prosthetic knee 100. The integral of active angular acceleration is likened to the angular velocity which may be used by the control algorithm to determine direction of the step and/or direction of rotation of the knee 100 (e.g., forward, backward, or lateral). In some embodiments, the IMU may additionally include a 3-axis magnetometer to detect additional positional information of the prosthetic knee 100.

Using measurements from the one or more inertial sensors and knee angle sensor described above, the control algorithm may instruct the microprocessor to adjust a valve assembly in the hydraulic damper 500 (as described in more detail below with respect to FIG. 6) in order to provide an appropriate flexion and/or extension resistance depending on the detected stage of gait. When the end user has the prosthesis loaded, the control algorithm may instruct the microprocessor to adjust the resistance of the hydraulic damper 500 to the stance flexion level 404 (e.g., the relatively higher flexion resistance level, for example shown in FIG. 4A, by activating a gearmotor 502 to adjust a position of a valve core 506), to provide sufficient support such that prosthetic knee 100 does not collapse underneath the user when loaded. When the user is ready to begin the swing phase (e.g., about to take a step), the control algorithm may instruct the microprocessor act to reduce the flexion resistance of the hydraulic damper 500 to a swing flexion level 402 (e.g., the relatively lower flexion resistance level, for example shown in FIG. 4A, by activating a gearmotor 502 to adjust a position of a valve core 506) in order to achieve heel rise. When the user is no longer achieving heel rise, the control algorithm may instruct the microprocessor to increase the flexion resistance back to the pre-set stance flexion level 404 to provide support again, and the cycle continues.

The control algorithm may only instruct the microprocessor to reduce the flexion resistance to the swing flexion level 402 on the occurrence of certain pre-conditions. In some embodiments, (e.g., embodiments using an IMU) there may be three pre-conditions, however more or fewer may also be used. A first pre-condition may be a detection, via measurements of the knee angle sensor, that the prosthetic knee 100 is fully or hyper extended. A second pre-condition may be a detection, via measurements of the gyroscope of the IMU, that the knee is not actively rotating backwards leading up to hyper-extension. A third pre-condition may be a detection, via measurements of the accelerometer of the IMU, of a forward tilt of the prosthetic knee 100. If any one of these pre-conditions is not true (e.g., present or met), the algorithm will instruct the microprocessor to maintain the prosthetic knee 100 at a stance flexion level 404 resistance until all three pre-conditions are satisfied. By design, these pre-conditions are typically only met at the Pre-Swing or Toe-Off phase of gait (e.g., Initial Swing phase or Mid-Swing phase shown in FIGS. 4A and 4B) when a user is walking forward.

In some embodiments, the algorithm may instruct the microprocessor to maintain swing flexion level 402 resistance if the gyroscope of the IMU indicates that the rotational velocity of the prosthetic knee 100 is at or above the threshold indicative of swing phase has been maintained.

In some embodiments, additional checks may also be used in order to detect specific edge cases where these pre-conditions may be satisfied at the wrong point of the gait cycle.

As discussed above, a user's initial vertical position may be calibrated when they are initially fitted with the prosthetic knee 100. The control algorithm may register the resistance level at the patient's initial vertical position as equivalent to the stance flexion level 404. As the user begins to walk, the control algorithm, using measurements from the knee angle sensor and the one or more inertial sensors, will monitor the stage of gait and instruct the microprocessor to adjust the flexion resistance accordingly.

The control algorithm may monitor the stage of gait based on the knee angle (determined based on signals detected by the knee angle sensor), the position, active rotation and direction of rotation of the prosthetic knee (determined based on signals detected by the one or more inertial sensors or IMU), and timing information. Timing information may include how long the prosthetic knee 100 has been in a particular stage of gait and/or a detected rate of change of rotation of the prosthetic knee 100.

The control algorithm may register that the user is transitioning from a first Stance phase to a first Swing phase upon a detection that a certain or pre-defined set of knee angle requirements, knee position requirements, and timing requirements are met (e.g., if the measured knee angle indicates the prosthetic knee 100 is fully or hyper extended, the knee is tilting forward or is vertical, the knee is not actively rotating backward, and the rate of change of the rotation is within a pre-determined range). In response to registering the user's transition from the first Stance phase to the first Swing phase (e.g., transitioning from Terminal Stance to Pre-Swing in FIG. 4A), the algorithm will instruct the microprocessor to lower the flexion resistance level (e.g., to swing flexion level 402). The control algorithm may register that the user is transitioning from the first Swing phase to a second Swing phase (e.g., transitioning from Pre-Swing to Initial Swing in FIG. 4A) upon a detection that a certain or pre-defined set of knee angle requirements, knee position requirements, and timing requirements are all met (e.g., if the measured knee angle indicates the prosthetic knee 100 is within a pre-determined threshold indicating the prosthetic knee 100 is fully flexed, is not actively rotating backward, and that the flexion has occurred within a pre-determined threshold amount of time). In response to registering the user's transition from the first Swing phase to the second Swing phase, the control algorithm will instruct the microprocessor to maintain the reduced flexion resistance level (e.g., swing flexion level 402).

The control algorithm may register that the user is transitioning from the second Swing phase back to the first Stance phase (e.g., as the knee transitions from Initial Swing to Mid-Swing in FIG. 4A) upon a detection that a certain pre-defined set of knee angle requirements are met (e.g., if the measured knee angle and position indicate that the knee 100 has transitioned from flexion to extension indicating impending heel strike or that the knee 100 has reached a threshold for full extension) or a detection that a certain or predefined set of timing requirements are met (e.g., if the knee 100 has been in this state for longer than a pre-determined threshold amount of time). In response to registering that the user is transitioning from the second Swing phase back to the first Stance phase, the control algorithm will instruct the microprocessor to increase the flexion resistance level (e.g., to stance flexion level 404).

In some instances, the user may be in the first Swing phase but not desire to actually take a step. This may occur, for example, when the user is walking or about to walk and has to stop suddenly. In such cases, the control algorithm may register that the user is transitioning from the first Swing phase back to the first Stance phase upon a detection that a certain or pre-defined set of knee angle requirements, knee position requirements, and knee timing requirements are met (e.g., if the measured knee angle and position indicate that the prosthetic knee 100 is not approaching full extension but has been in the first Swing phase for longer than a pre-determined threshold amount of time), or upon a detection that a certain or pre-defined set of knee angle requirements and knee timing requirements are met (e.g., if the measured knee angle indicates that the prosthetic knee 100 is not approaching full extension, and the rotation speed is below a certain pre-defined threshold). In response to registering the user's transition from the first Swing phase to the first Stance phase, the control algorithm will instruct the microprocessor to increase the flexion resistance level (e.g., to stance flexion level 404).

In some instances, the user may want to step or walk backwards. The control algorithm may register that the user is transitioning from the first Stance phase to a second Stance phase upon a detection that a certain or pre-defined set of knee angle requirements, knee position requirements, and knee timing requirements are met (e.g., if the measured knee angle and position indicate that the prosthetic knee 100 is extended, tilted forward, and rotating backward within a pre-determined time range). In response to registering the user's transition from the first Stance phase to the second Stance phase, the control algorithm will instruct the microprocessor to maintain the flexion resistance level at stance flexion level 404. The control algorithm may register that the user is transitioning from the second Stance phase to the first Stance phase upon a detection that a certain or pre-defined set of knee position requirements are met (e.g., if the measured knee position indicates that the prosthetic knee 100 is not currently rotating backward, and is either vertical or tilting backward (e.g., dorsal relative to the axial pin shaft 202)). In response to registering the user's transition from the second Stance phase to the first Stance phase, the control algorithm will instruct the microprocessor to maintain the flexion resistance level at stance flexion level 404.

In some embodiments, as shown in FIG. 4B, the control algorithm may instruct the microprocessor to maintain a high flexion resistance level 406 as a default control in order to support the user in case they stumble during gait (e.g., if they stumble during the transition from Initial Swing to Mid-Swing in FIG. 4A). The high flexion resistance level 406 may be higher than the stance flexion level 404 set by the CPO based on the end user's needs when the user is initially fitted with the prosthetic knee 100. The control algorithm may instruct the microprocessor to provide a high flexion resistance level 406 during swing extension and then transition to the stance flexion level 404 upon a detection by the knee angle sensor that the knee 100 has reached a certain, pre-defined threshold angle (e.g., during Terminal Swing). In some embodiments, the threshold angle may be between 0 degrees and 10 degrees (e.g., 1°, 2°, 3°, 4°, 5°, 6°, 7°, 8°, 9°, ranges between such values, and the like, etc.). This may advantageously improve stumble recovery by providing even more support to the user regardless of their preferred stance flexion resistance (e.g., stance flexion level 404) should they stumble.

In other embodiments, the control algorithm may implement a stumble recovery adjustment in order to support the user in case they stumble during gait, rather than maintaining high flexion resistance level 406 as a default control. The control algorithm may instruct the microprocessor to implement the a stumble recovery adjustment to provide a high flexion resistance level 406 upon a detection that a certain or pre-defined set of knee angle requirements and knee timing requirements are met (e.g., if the measured knee angle exceeds a pre-defined threshold value, and the measured knee angle rate of change is less than a pre-determined threshold value) as the user is transitioning from the second Swing phase to the first Stance Phase (e.g., as the knee transitions from Initial Swing to Mid-Swing in FIG. 4B). The control algorithm may instruct the microprocessor to exit the stumble recovery phase and reduce the flexion level from high flexion resistance level 406 back to stance flexion level 404 upon a detection that the prosthetic knee 100 has successfully transitioned to the first or second Stance phase, and/or that that a certain or pre-defined set of knee position requirements and knee angle requirements are met (e.g., if the measured knee position indicates that the knee is fully extended, and the measured knee angle is at or below a pre-determined threshold value). This may advantageously improve stumble recovery by providing even more support to the user regardless of their preferred stance flexion resistance (e.g., stance flexion level 404) should they stumble.

In some embodiments, the control algorithm described herein may be capable of recognizing when the user is descending stairs using measurements from the knee angle sensor and the one or more inertial sensors. When motion indicative of stair descent occurs, the control algorithm may instruct the microprocessor to maintain a high resistance level (e.g., stance flexion level 404 in FIG. 4A) such that transitioning to the first Swing phase, or the second Swing phase is not possible. This advantageously allows the knee to maintain a higher resistance to support the end user and allows them to ride the knee during stair descent. Additionally, it will also be possible to provide an increased stance flexion level resistance 404 at the moment stair descent is detected.

Use of a control algorithm to adjust the flexion resistance of the prosthetic knee 100 during gait, as described above is advantageous for several reasons. For example, because the control algorithm only requires measurements from a single inertial sensor/IMU and the knee position sensor (e.g., knee angle sensor), this control scheme eliminates the need for additional sensors (e.g., torque sensors, load cells, etc.) advantageously reducing the weight of the prosthetic knee 100. Reducing the number of sensors also results in less complicated processing, allowing the microprocessor to make decisions more quickly. Requiring fewer sensors additionally reduces the power requirements of the prosthetic knee 100 improving battery life. In some embodiments, the prosthetic knee 100 may operate using a single-cell 18650 battery.

Further, this control scheme advantageously does not require unnecessary patient specific information, such as foot size, height, weight, etc. in order to operate. For example, the control algorithm described herein does not require multiple operating states (e.g., ramp mode, stairs mode) or specific identification of a user's ADL's. The control algorithm described herein also does not require measurement of angles based on center of mass and chord length of the prosthesis in order to operate.

In some embodiments, the control algorithm described herein can additionally or alternatively be used to automatically adjust an extension resistance of the prosthetic knee 100 during gait, as shown in FIG. 5. For example, in instances where the control algorithm registers the user's transition from the first Stance phase to the first Swing phase, the algorithm may instruct the microprocessor to decrease the extension resistance level from a stance extension resistance level 604 to a swing extension resistance level 602 (e.g., as shown in FIG. 5, by activating a gearmotor 504 to adjust a position of a valve core 508). Additionally, or alternatively, the control algorithm may instruct the microprocessor to decrease the extension resistance level to swing extension level 602 upon a detection that the measured knee angle exceeds a pre-determined threshold value while a high extension resistance level (e.g., stance extension resistance level 604) is being applied to the prosthetic knee 100. The control algorithm may instruct the microprocessor to increase the extension resistance from swing extension resistance level 602 to stance extension resistance level 604 in response to a detection that the user is transitioning from the second Swing phase to the first Stance phase, and that the measured knee angle is below a pre-determined threshold value.

FIG. 6 shows a front view of an example hydraulic damper 500 of the prosthetic knee 100. The hydraulic damper 500 may include a cylinder 505, a piston 510, hydraulic fluid (e.g., glycol ether, organophosphate ester, polyalphaolefin, a propylene glycol, a silicone oil, NaK-7, etc.), and a valve-gear motor system. The piston 510 may move slidably along the length of the cylinder 505 in response to a change in the amount of hydraulic fluid within the cylinder, which is controlled by the valve-gear motor system. Gearmotors 502 and 504 are activated in response to feedback from the knee angle sensor and accordingly rotate the valve cores 506 and 508, respectively, of valve cartridges (where each valve cartridges can include a valve sleeve and a valve core). Motor couplers 507 and 509 connect gearmotors 502 and 504 and valve cores 506 and 508, respectively. As the valve cores 506, 508 are rotated by the gearmotors 502, 504 via the motor couplers 507, 509 based on information from the knee angle sensor and other sensors of the prosthetic knee 100 that provide information indicative of the user's gait, the size of a valve orifice in their respective valve cartridges via which hydraulic fluid passes can be varied, which in turn changes the hydraulic resistance by exerting forces on the piston 510.

In some embodiments, the resistances for the bending and stretching (i.e., flexion and extension) motions of the prosthetic knee 100 may be set separately by individual valve and gearmotor assemblies. For example, valve core 506 may be controlled by gearmotor 502 to adjust the level of flexion resistance while valve core 508 may be controlled by gearmotors 504 to adjust the level of extension resistance, or vice versa. For example, when the knee angle is registered as being fully extended (e.g., at 0°-2° of flexion) by the knee angle sensor, the microprocessor will verify with the inertial sensor(s) or IMU that the lower frame portion 104 of the prosthetic knee 100 is also tilted and/or actively rotating forward (e.g., when fully extended and behind the user). This is indicative of a “toe-off” condition. The microprocessor may then command the gearmotor 502 to rotate valve core 506 (e.g., the flexion valve) to a pre-set lower resistance (e.g., swing flexion) allowing the user to achieve heel rise.

The knee angle sensor continuously monitors the knee angle and changes in the knee angle. Once the knee angle sensor detects that the knee angle is no longer increasing (i.e., flexing) in swing phase and the knee angle is extending again, the microprocessor will command the gearmotor 502 to rotate the valve core 506 (e.g., the flexion valve) back to the pre-set stance flexion resistance to prepare for heel strike. If the lower frame portion 104 of the prosthetic knee 100 has been detected as being tilted backwards while the knee angle is fully extended, this is indicative of the heel strike position, and the stance flexion resistance settings will remain. If the prosthetic knee 100 is flexed beyond a fully extended threshold, it will maintain its pre-set stance flexion resistance and will not go into a lower swing flexion resistance even if the prosthetic knee 100 is tilted forwards. Furthermore, to ensure that changes to the valve resistances are only being made when the user is actively walking, the knee angle sensor and one or more inertial sensors will monitor for change in the knee angle and position before changing resistance settings via the valve cores 506, 508. This advantageously prevents the prosthetic knee 100 from going into a lower resistance state on a false step. Alternate means of correlating the knee angle position to gait patterns may also be used, such as, for example, a look-up table.

Various modifications to the embodiments described in this disclosure will be readily apparent to those skilled in the art, and the generic principles defined herein may be applied to other embodiments without departing from the spirit or scope of this disclosure. Thus, the disclosure is not intended to be limited to the embodiments discussed herein but is to be accorded the widest scope consistent with the claims, the principles and the novel features disclosed herein. The word “example” is used exclusively herein to mean “serving as an example, instance, or illustration.” Any embodiment described herein as “example” is not necessarily to be construed as preferred or advantageous over other embodiments, unless otherwise stated.

Certain features that are described in this specification in the context of separate embodiments also may be embodied in combination in a single embodiment. Conversely, various features that are described in the context of a single embodiment also may be embodied in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the order shown or in sequential order, or that all illustrated operations be performed, to achieve desirable results. Additionally, other embodiments are within the scope of the following claims. In some cases, the actions recited in the claims may be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

What is claimed is:

1. A prosthetic joint, comprising:
a frame;
a connector pivotally coupled to a proximal portion of the frame, the connector configured to pivot in an anterior-posterior direction of the frame about a pivot axis that extends in a medial-lateral direction of the frame, the frame and connector being portions of a joint;
a shaft extending along the pivot axis, the shaft extending through the connector and coupled to the frame on opposite sides of the connector;
an inertial sensor attached to the frame,
a knee angle sensor attached to the frame at or proximate the pivot axis; and
a microprocessor configured to receive signals detected by the inertial sensor and the knee angle sensor to detect a gait event and to automatically adjust a flexion resistance and/or an extension resistance of the prosthetic joint in response to the detected gait event;
wherein the flexion resistance and/or the extension resistance of the prosthetic joint is controlled solely by information from the signals for the gait event detected by the knee angle sensor and the inertial sensor.

2. The prosthetic joint of claim 1, wherein the prosthetic joint is a prosthetic knee.

3. The prosthetic joint of claim 2, wherein the microprocessor adjusts the flexion resistance to a lower resistance level in response to the signals from the inertial sensor indicating the prosthetic joint is fully or hyper-extended, there is not an active backward rotation of the prosthetic joint, and there is a forward tilt of the prosthetic knee.

4. The prosthetic joint of claim 2, wherein the microprocessor adjusts the flexion resistance to a lower resistance level only when:
a detection is made by the knee angle sensor that the prosthetic joint is fully or hyper-extended;
a detection is made by the inertial sensor that there is an active forward rotation of the prosthetic joint; and
a detection is made by the inertial sensor that there is a forward tilt of the prosthetic knee.

5. The prosthetic joint of claim 2, wherein the prosthetic knee includes a hydraulic damper including a cylinder and piston configured for slidable travel within the cylinder.

6. The prosthetic joint of claim 5, wherein the microprocessor is configured to control an operation of the hydraulic damper in order to adjust a flexion resistance of the prosthetic joint.

7. The prosthetic joint of claim 1, wherein the inertial sensor is an inertial measurement unit (IMU).

8. The prosthetic joint of claim 7, wherein the IMU comprises one or more of:
an accelerometer;
a gyroscope; and/or
a magnetometer.

9. The prosthetic joint of claim 1, further comprising:
a diametrically polarized magnet housed in a magnet cup, the magnet cup being housed in a hollow portion of the shaft so that the magnet is centered along the pivot axis, the magnet cup separating the magnet from the shaft,
wherein rotation of the connector relative to the frame simultaneously rotates the shaft, the magnet cup, and the diametrically polarized magnet, and wherein the knee angle sensor is configured to measure a motion of the connector relative to the shaft by detecting a magnetic field generated by the magnet.

10. The prosthetic joint of claim 9, wherein the magnet cup is configured to separate the diametrically polarized magnet from the shaft to not inhibit a magnetic field output of the diametrically polarized magnet.

11. The prosthetic joint of claim 1, wherein the inertial sensor is attached to first circuit board.

12. The prosthetic joint of claim 1, wherein the knee angle sensor is attached to a second circuit board.

13. The prosthetic joint of claim 12, wherein the knee angle sensor is encased in a coating and/or an overmold and wherein the coating and/or the overmold is configured to electrically isolate the second circuit board.

14. The prosthetic joint of claim 1, wherein the microprocessor is configured to correlate the signals from the knee angle sensor to a knee angle measurement.

15. A method for controlling a prosthetic joint, the method comprising:
measuring, by an inertial sensor, a first set of parameters of the prosthetic joint;
measuring, by a knee angle sensor, a second set of parameters of the prosthetic joint;
receiving, by a microprocessor, signals representing the first and second set of parameters;
determining, by the microprocessor, a level of flexion resistance and/or extension resistance to be applied to the prosthetic joint based on the signals;
detecting, by the microprocessor, a gait event based on the received signals; and
adjusting, by the microprocessor, a resistance of the prosthetic joint to the level of flexion resistance and/or extension resistance for the detected gait event solely based on the signals.

16. The method of claim 15, wherein the prosthetic joint is a prosthetic knee.

17. The method of claim 16, wherein the determining comprises determining that one or more preconditions exist.

18. The method of claim 17, wherein the one or more preconditions include one or more of:
a detection that the prosthetic joint is fully or hyper-extended;
a detection that there is an active forward rotation of the prosthetic joint; and
a detection that there is a forward tilt of the prosthetic knee.

19. The method of claim 17, wherein the one or more preconditions include one or more of:
a detection by the knee angle sensor that the prosthetic joint is fully or hyper-extended;
a detection by the inertial sensor that there is an active forward rotation of the prosthetic joint; and
a detection by the inertial sensor that there is a forward tilt of the prosthetic knee.

20. The method of claim 17, further comprising:
determining that at least one of the one or more preconditions do not exist; and
adjusting, by the microprocessor, the flexion resistance to a first level.
